(19) United States
(12) Patent Application Publication
(10) Pub. No.: US 2018/0030093 A1
(43) Pub. Date: Feb. 1, 2018

(54) SELF-ASSEMBLING ULTRASHORT ALIPHATIC CYCLIC PEPTIDES FOR BIOMEDICAL APPLICATIONS

(71) Applicant: Agency for Science, Technology and Research, Singapore (SG)

(72) Inventors: Charlotte A. E. Hauser, Singapore (SG); Michael R. Reithofer, Singapore (SG)

(21) Appl. No.: 15/551,116

(22) PCT Filed: Mar. 31, 2016
(86) PCT No.: PCT/SG2016/050159

§ 371 Date: Aug. 15, 2017

(30) Foreign Application Priority Data
Mar. 31, 2015 (SG) 10201502526Y

(51) Int. Cl. C07K 7/64; A61K 8/64; A61K 9/00; A61K 9/06; A61K 8/04; A61K 47/42; A61Q 19/00

(52) U.S. Cl. CPC C07K 7/64; A61K 47/42; A61K 8/64; A61Q 19/00; A61K 9/06; A61K 8/042; A61K 9/0019; A61K 2800/10; A61K 2800/91

(57) ABSTRACT

The invention relates to cyclic peptides of 3-9 amino acids comprising 2-7 aliphatic and 0-2 polar amino acids that are capable of self-assembling, wherein said aliphatic amino acids are arranged in decreasing hydrophobicity from N- to C-terminus and at least a portion of the cyclic peptide has to have its amino acids in alternating D- and L-configuration, as well as their use in hydrogels as well as co-gels or co-hydrogels. The hydrogels of the invention may be used in nanomedicine or drug delivery, cell culture or alternatively in electronic devices.

### FIELD OF INVENTION

The present invention is in the area of nanomedicine and drug delivery. The invention generally relates to cyclic peptides and their use in hydrogels as well as in co-gels or co-hydrogels.

### BACKGROUND

The following discussion of the background to the invention is intended to facilitate an understanding of the present invention. However, it should be appreciated that the discussion is not an acknowledgment or admission that any of the material referred to was published, known or part of the common general knowledge in any jurisdiction as at the priority date of the application.

The microarchitecture of nanofibrous hydrogels made ultrashort peptides (see e.g. Hauser et al., 2011) can resemble extracellular matrix, opening avenues for widespread applications as biomimetic scaffolds for tissue engineering and three-dimensional cell culture. Furthermore, such hydrogels demonstrate remarkable mechanical stiffness, thermostability, biocompatibility, in vitro and in vivo stability. However, in developing such hydrogels for shorter-term applications such as injectable matrices for drug and gene delivery, it is desirable to precisely control the drug release rate.

Although the self-assembling properties of cyclic peptides are well known (Mandal et al., 2014; Montenegro et al., 2013; Li et al., 2012), most of the reported systems do not form hydrogels. Hydrogel formation could so far only be achieved using rigid structures. Recently several groups independently report on the hydrogel formation using functionalized cyclic dipeptides. However, a cyclic dipeptide not only represents the smallest possible cyclic peptide, but is often better described as a diketopiperazine unit, and can thus be not considered as a macrocyclic peptide. Gelation of diketopiperazine is achieved through additional functionalization of the amino acid side chain and cannot be seen as an intrinsic molecular behaviour (Manchineella and Govindaraju, 2012; Hoshizawa et al., 2013; Kleinsmann and Nachtsheim, 2013).

Thus, there is a need in the art of nanomedicine for improved means and methods for controlled release or delivery of (bioactive) compounds.

### SUMMARY

The present technology proposes ultrashort aliphatic cyclic peptides which are capable of self-assembling into hydrogels. The present invention comprises the following features:

Key technical features:
Use of the class of ultrashort peptides, as described before by the inventors, which are cyclized through a head to tail cyclization reaction.
Ability of these molecules to self-assemble in water to form hydrogels.
Ability of these compounds to self-assemble into hydrogels made of nanotubes.
Development of nano-tubular hydrogels, for example, for drug delivery.

This disclosure describes a technology to synthesize ultrashort aliphatic cyclic peptides, which are capable of self-assembly, into hydrogels made of nanotubes in aqueous conditions. The synthesized cyclic peptides are able to form into hydrogels with low peptide content (as low as 5 mg/mL).

The cyclic peptides can also be mixed with the parent ultrashort peptide to create co-gels, for adjustments of mechanic properties, for example, release profile and solubility.

In accordance with an aspect of the present invention, the invention provides a cyclic peptide and/or peptidomimetic capable of self-assembling and forming a hydrogel in aqueous solutions, the cyclic peptide and/or peptidomimetic having the general formula:

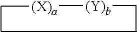

wherein
X is, at each occurrence, independently selected from the group consisting of aliphatic amino acids and aliphatic amino acid derivatives, and wherein the overall hydrophobicity decreases from N- to C-terminus;
a is an integer selected from 2 to 7;
Y is selected from the group consisting of polar amino acids and polar amino acid derivatives;
b is 0, 1 or 2;
and a+b is at least 3.

In one embodiment, all or a portion of said aliphatic amino acids and aliphatic amino acid derivatives, and said polar amino acids and polar amino acid derivatives alternate with respect to L-amino acids and D-amino acids,

i.e. after an L-amino acid follows an D-amino acid which is followed by an L-amino acid and so on.

In one embodiment, said aliphatic amino acids are selected from the group consisting of alanine (Ala, A), homoallylglycine, homopropargylglycine, isoleucine (Ile, I), norleucine, leucine (Leu, L), valine (Val, V) and glycine (Gly, G),

preferably from the group consisting of alanine (Ala, A), isoleucine (Ile, I), leucine (Leu, L), valine (Val, V) and glycine (Gly, G).

In one embodiment, all or a portion of said aliphatic amino acids are arranged in an order of decreasing amino acid size, wherein the size of the aliphatic amino acids is defined as I=L>V>A>G.

In one embodiment, (X)ₐ has a sequence selected from

(SEQ ID NO: 1)
LIVAG,
(SEQ ID NO: 2)
ILVAG,
(SEQ ID NO: 3)
LIVAA,
(SEQ ID NO: 4)
LAVAG,
(SEQ ID NO: 5)
IVAG
(SEQ ID NO: 6)
LVAG,
(SEQ ID NO: 7)
ILV,
(SEQ ID NO: 8)
LIVA
(SEQ ID NO: 9)
LIVG
IVG,
VIG,
IVA,
VIA,
IV,
IL,
LV,
VA,
VG,
IG,
IA,
and
LA

wherein, optionally, there is an G, V or A preceding such sequence at the N-terminus, such as

(SEQ ID NO. 10)
AIVAG,
(SEQ ID NO. 11)
GIVAG,
(SEQ ID NO. 12)
VIVAG,
(SEQ ID NO. 13)
ALVAG,
(SEQ ID NO. 14)
GLVAG,
(SEQ ID NO. 15)
VLVAG.

In one embodiment, a is an integer from 3 to 7, 3 to 6 or 2 to 6,

or more preferably 3 to 5.

In one embodiment, said polar amino acids are selected from the group consisting of aspartic acid (Asp, D), asparagine (Asn, N), glutamic acid (Glu, E), glutamine (Gln, Q), 5-N-ethyl-glutamine (theanine), citrulline, thio-citrulline, cysteine (Cys, C), homocysteine, methionine (Met, M), ethionine, selenomethionine, telluromethionine, threonine (Thr, T), allothreonine, serine (Ser, S), homoserine, arginine (Arg, R), homoarginine, ornithine (Orn), lysine (Lys, K), N(6)-carboxymethyllysine, histidine (His, H), 2,4-diaminobutyric acid (Dab), 2,3-diaminopropionic acid (Dap), and N(6)-carboxymethyllysine,

wherein said polar amino acid is preferably selected from the group consisting of aspartic acid, asparagine, glutamic acid, glutamine, serine, threonine, methionine, lysine, ornithine (Orn), 2,4-diaminobutyric acid (Dab), and 2,3-diaminopropionic acid (Dap).

In one embodiment,
b is 2 and said polar amino acids are identical amino acids, or
b is 1 and said polar polar amino acid comprises any one of aspartic acid, asparagine, glutamic acid, glutamine, serine, threonine, cysteine, methionine, lysine, ornithine, 2,4-diaminobutyric acid (Dab) and histidine,
preferably lysine, ornithine, 2,4-diaminobutyric acid (Dab) and 2,3-diaminopropionic acid (Dap).

In one embodiment, (Y)_{b} has a sequence selected from Asp, Asn, Glu, Gln, Ser, Thr, Cys, Met, Lys, Orn, Dab, His, Asn-Asn, Asp-Asp, Glu-Glu, Gln-Gln, Asn-Gln, Gln-Asn, Asp-Gln, Gln-Asp, Asn-Glu, Glu-Asn, Asp-Glu, Glu-Asp, Gln-Glu, Glu-Gln, Asp-Asn, Asn-Asp Thr-Thr, Ser-Ser, Thr-Ser, Ser-Thr, Asp-Ser, Ser-Asp, Ser-Asn, Asn-Ser, Gln-Ser, Ser-Gln, Glu-Ser, Ser-Glu, Asp-Thr, Thr-Asp, Thr-Asn, Asn-Thr, Gln-Thr, Thr-Gln, Glu-Thr, Thr-Glu, Cys-Asp, Cys-Lys, Cys-Ser, Cys-Thr, Cys-Orn, Cys-Dab, Cys-Dap, Lys-Lys, Lys-Ser, Lys-Thr, Lys-Orn, Lys-Dab, Lys-Dap, Ser-Lys, Ser-Orn, Ser-Dab, Ser-Dap, Orn-Lys, Orn-Orn, Orn-Ser, Orn-Thr, Orn-Dab, Orn-Dap, Dab-Lys, Dab-Ser, Dab-Thr, Dab-Orn, Dab-Dab, Dab-Dap, Dap-Lys, Dap-Ser, Dap-Thr, Dap-Orn, Dap-Dab, Dap-Dap.

In one embodiment, (X)ₐ-(Y)_{b} has a sequence selected from the group consisting of

(SEQ ID NO: 16)
LIVAGK,
(SEQ ID NO. 17)
ILVAGK,
(SEQ ID NO: 18)
LIVAAK,
(SEQ ID NO: 19)
LAVAGK,
(SEQ ID NO: 20)
AIVAGK,
(SEQ ID NO: 21)
LIVAGS,
(SEQ ID NO. 22)
ILVAGS,
(SEQ ID NO: 23)
LIVAAS,
(SEQ ID NO: 24)
LAVAGS,
(SEQ ID NO: 25)
AIVAGS,
(SEQ ID NO: 26)
LIVAGD,
(SEQ ID NO: 27)
ILVAGD,
(SEQ ID NO: 28)
LIVAAD,
(SEQ ID NO: 29)
LAVAGD,
(SEQ ID NO: 30)
AIVAGD,
(SEQ ID NO: 31)
LIVAGE,
(SEQ ID NO: 32)
LIVAGT,
(SEQ ID NO: 33)
ILVAGT.
(SEQ ID NO: 34)
AIVAGT,
(SEQ ID NO: 35)
AIVAGK,
(SEQ ID NO: 36)
LIVAD,
(SEQ ID NO: 37)
LIVGD,
(SEQ ID NO: 38)
IVAD,
(SEQ ID NO: 39)
IVAK,
(SEQ ID NO: 40)
LIVAGOrn,
(SEQ ID NO: 41)
ILVAGOrn,
(SEQ ID NO: 42)
AIVAGOrn,
(SEQ ID NO: 43)
LIVAGDab,
(SEQ ID NO: 44)
ILVAGDab,
(SEQ ID NO: 45)
AIVAGDab,
(SEQ ID NO: 46)
LIVAGDap,
(SEQ ID NO: 47)
ILVAGDap,
(SEQ ID NO: 48)
AIVAGDap,
(SEQ ID NO: 49)
LIVAGKK,
(SEQ ID NO: 50)
LIVAGSS,
(SEQ ID NO: 51)
LIVAGDD,
(SEQ ID NO: 52)
LIVAGEE,
IVD,
LVD,
IAK,
IVK,
LVK,
and
VAK,

such as LIVAGK (SEQ ID NO: 16)
   (with L and V being D-amino acids and I, A, and K being L-amino acids)
   LIVAGS (SEQ ID NO: 21)
   (with L and V being D-amino acids and I, A, and K being L-amino acids).

In one embodiment, a+b is at least 3, preferably 3 to 6 or 4 to 6, more preferably 6.

In one embodiment, the peptides are cyclized via head-to-tail cyclization.

In one embodiment, said cyclic peptides self-assemble in aqueous solution to form hydrogels, preferably hydrogels made of nanotubes or nanocontainers.

Preferably, the cyclic peptides are stacked during self-assembly and, thus, form nanotubes or nanocontainers.

Preferably, self-assembly is achieved through non-covalent interaction.

In one embodiment, said cyclic peptides are stable in aqueous solution at physiological conditions at ambient temperature for a period of time in the range from 1 day to at least 6 months, preferably to at least 8 months more preferably to at least 12 months.

In one embodiment, said cyclic peptides are stable in aqueous solution at physiological conditions, at a temperature up to 90° C., for at least 1 hour.

In accordance with an aspect of the present invention, the invention provides the use of a cyclic peptide according to the present invention:
as β-sheet breaker;
as anti-microbial agent or compound;
for encapsulating active compounds and/or cells through non-covalent interaction;
for drug delivery;
for nano printing;
as nano template for nano wires;
as additive in other peptide-based hydrogels;
as channel pores in membranes.

In accordance with an aspect of the present invention, the invention provides a method of preparing a hydrogel, the method comprising dissolving at least one cyclic peptide of the present invention in an aqueous solution.

In one embodiment, the at least one cyclic peptide is dissolved at a concentration from about 0.01 μg/ml to 100 mg/ml, preferably at a concentration from 1 mg/ml to 50 mg/ml, more preferably at a concentration from 5 mg/mL to 15 mg/mL or 5 mg/mL to 10 mg/mL.

In one embodiment, the dissolved cyclic peptide and/or peptidomimetic in aqueous solution is further exposed to temperature, wherein the temperature is in the range from 20° C. to 90° C., preferably from 20° C. to 70° C., such as about 60° C.

In one embodiment, the method comprises the dissolution of the cyclic peptide in an organic solvent and subsequently dropwise addition into an aqueous solution, such as water.

In one embodiment, the method comprises the addition of further compound(s) prior or during gelation/self-assembly, which are encapsulated by the hydrogel,
wherein said further compound(s) can be selected from
   bioactive molecules or moieties,
      such as growth factors, cytokines, lipids, cell receptor ligands, hormones, prodrugs, drugs, vitamins, antigens, antibodies, antibody fragments, oligonucleotides (including but not limited to DNA, messenger RNA, short hairpin RNA, small interfering RNA, microRNA, peptide nucleic acids, aptamers), saccharides;
   label(s), dye(s),
      such as imaging contrast agents;
   pathogens,
      such as viruses, bacteria and parasites;
   quantum dots, nano- and microparticles,
   or combinations thereof.

In one embodiment, the method comprises the addition or mixing of cells prior or during gelation/self-assembly, which are encapsulated by the hydrogel,
wherein said cells can be stem cells (mesenchymal, progenitor, embryonic and induced pluripotent stem cells), transdifferentiated progenitor cells and primary cells isolated from patient samples (fibroblasts, nucleus pulposus).

preferably comprising the addition of further compound(s) prior or during gelation (such as defined in claim 21), which are co-encapsulated by the hydrogel,

optionally comprising the addition or mixing of different cells prior or during gelation/self-assembly and/or comprising the addition or mixing of cells onto the hydrogel after gelation.

Preferably in this embodiment, the method comprises the following steps:

(1) the addition or mixing of cells prior or during gelation, which are encapsulated by the hydrogel, and

(2) subsequently the addition of cells onto the printed hydrogel,

wherein said cells of (1) and (2) are the same or different,

and can be stern cells (adult, progenitor, embryonic and induced pluripotent stern cells), transdifferentiated progenitor cells, and primary cells (isolated from patients) and cell lines (such as epithelial, neuronal, hematopoietic and cancer cells).

In one embodiment, the method comprises the use of different cyclic peptides.

In accordance with an aspect of the present invention, the invention provides a method of preparing a co-gel or co-hydrogel, the method comprising

(a) dissolving at least one cyclic peptide of the present invention in an aqueous solution,

(b) dissolving at least one peptide which has the same sequence as the cyclic peptide of step (a), but includes only L-amino acids or only D-amino acids (“parent peptide”), in an aqueous solution,

(c) mixing the solutions of (a) and (b) and gelating,

(d) obtaining the co-gel or co-hydrogel.

In accordance with an aspect of the present invention, the invention provides a hydrogel comprising at least one cyclic peptide of the present invention,

preferably obtained by a method of the present invention.

In one embodiment, the hydrogel is stable in aqueous solution at ambient temperature for a period of at least 7 days, preferably at least 2 to 4 weeks, more preferably at least 1 to 6 months.

In one embodiment, the hydrogel is characterized by a storage modulus G′ to loss modulus G″ ratio that is greater than 2.

In one embodiment, the hydrogel is characterized by a storage modulus G′ from 100 Pa to 80,000 Pa at a frequency in the range of from 0.02 Hz to 16 Hz.

In accordance with an aspect of the present invention, the invention provides a co-gel or co-hydrogel comprising

at least one cyclic peptide of the present, and

at least one parent peptide, i.e. a peptide which has the same sequence as the cyclic peptide, but includes only L-amino acids or only D-amino acids,

preferably obtained by the method of preparing a co-gel or co-hydrogel of the present invention, as described above.

In one embodiment, the co-gel or co-hydrogel is adjusted with regard to its mechanical properties, such as release profile and/or solubility,

compared to the hydrogel comprising only the parent peptide, i.e. the peptide which has the same sequence as the cyclic peptide but includes only L-amino acids or only D-amino acids, and not the cyclic peptide.

In one embodiment, hydrogel of the present invention or the co-gel or co-hydrogel of the present invention furthermore comprise:
further compound(s), which are encapsulated by the hydrogel or the co-gel or co-hydrogel, wherein said further compound(s) can be selected from
   bioactive molecules or moieties,
      such as growth factors, cytokines, lipids, cell receptor ligands, hormones, prodrugs, drugs, vitamins, antigens, antibodies, antibody fragments, oligonucleotides (including but not limited to DNA, messenger RNA, short hairpin RNA, small interfering RNA, microRNA, peptide nucleic acids, aptamers), saccharides;
   label(s), dye(s),
      such as imaging contrast agents;
   pathogens,
      such as viruses, bacteria and parasites;
   quantum dots, nano- and microparticles,
   or combinations thereof;

and/or
cells, which are encapsulated by the hydrogel or the co-gel or co-hydrogel and/or added onto the hydrogel or the co-gel or co-hydrogel after gelation

wherein said cells are the same or different, and can be stem cells (adult, progenitor, embryonic and induced pluripotent stern cells), transdifferentiated progenitor cells, and primary cells (isolated from patients) and cell lines (such as epithelial, neuronal, hematopoietic and cancer cells).

In accordance with an aspect of the present invention, the invention provides the use of the hydrogel of the present invention or the co-gel or co-hydrogel of the present invention:
for encapsulating further compound(s) and/or cells through non-covalent interaction;
3D cell culture;
for drug delivery, in particular for sustained release;
for nano printing, preferably with cells;
as nano template for nano wires,
   such as for templating metal, ceramic, silicate and/or semiconductor nanotubes;
as pores or channels in membranes.

In accordance with an aspect of the present invention, the invention provides a pharmaceutical and/or cosmetic composition comprising

at least one cyclic peptide of the present invention,

a hydrogel of the present invention,

or

a co-gel or co-hydrogel of the present invention.

In one embodiment, the pharmaceutical and/or cosmetic of the present invention further comprises a pharmaceutically active compound, and optionally a pharmaceutically acceptable carrier.

In one embodiment, the pharmaceutical and/or cosmetic composition is injectable.

In accordance with an aspect of the present invention, the invention provides a biomedical devive comprising

at least one cyclic peptide of the present invention,

a hydrogel of the present invention,

or

a co-gel or co-hydrogel of the present invention.

In one embodiment, the biomedical device of the present invention further comprises a pharmaceutically active compound, and optionally a pharmaceutically acceptable carrier.

In accordance with an aspect of the present invention, the invention provides a surgical implant comprising

at least one cyclic peptide of the present invention,

a hydrogel of the present invention,

or

a co-gel or co-hydrogel of the present invention.

In one embodiment, the surgical implant of the present invention further comprises a pharmaceutically active compound, and optionally a pharmaceutically acceptable carrier.

In accordance with an aspect of the present invention, the invention provides an electronic device comprising

at least one cyclic peptide of the present invention,

a hydrogel of the present invention,

or

a co-gel or co-hydrogel of the present invention.

optionally, metal, ceramic, silicate and/or semiconductor nanotubes.

In accordance with an aspect of the present invention, the invention provides a kit of parts, the kit comprising

a first container with at least one cyclic peptide of the present invention, and

a second container with an aqueous solution,
wherein optionally the first and/or second contained further comprises a pharmaceutically active compound,

In one embodiment, the kit of parts further comprises

a fourth container with at least one parent peptide of the at least one cyclic peptide of the first container, and

a fifth container with an aqueous solution.

In one embodiment, at least one of said first, second , third, fourth or fifth container is provided as a spray bottle or a syringe.

In accordance with an aspect of the present invention, the invention provides the use of
a cyclic peptide of the present invention,
a hydrogel of the present invention,
a co-gel or co-hydrogel of the present invention, or
a pharmaceutical and/or cosmetic composition and/or a biomedical device and/or a surgical implant of the present invention, for:
   regenerative medicine and tissue regeneration or tissue replacement,
      e.g. regeneration of adipose and cartilage tissue,
   implantable scaffold
   disease model
   wound treatment and/or wound healing,
   2D and 3D synthetic cell culture substrate,
   stem cell therapy,
   drug delivery, preferably sustained or controlled release drug delivery
   injectable therapies,
   treatment of degenerative diseases of the skeletal system,
      e.g. degenerative disc disease, or urinary incontinence
   biosensor development,
   high-throughput screening,
   biofunctionalized surfaces,
   biofabrication, such as bioprinting,
   cosmetic use;
   and
   gene therapy.

In accordance with an aspect of the present invention, the invention provides a method of tissue regeneration or tissue replacement comprising the steps:
a) providing a hydrogel according to the present invention, or a co-gel or co-hydrogel according to the present invention;
b) exposing said hydrogel or co-gel or co-hydrogel to cells which are to form regenerated tissue;
c) allowing said cells to grow on or in said hydrogel.

In one embodiment, the method is performed in vitro or in vivo or ex vivo.

Preferably, the method is performed in vivo, wherein, in step a), said hydrogel or co-gel or co-hydrogel is provided at a place in the body of a patient where tissue regeneration or tissue replacement is intended.

In one embodiment, said step a) is performed by injecting said or co-gel or co-hydrogel or a solution of at least one cyclic peptide of the present invention, at a place in the body of a patient where tissue regeneration or tissue replacement is intended.

Preferably, the method is performed ex vivo, wherein, in step a) or b), cells from a patient or from a donor are mixed with said hydrogel or co-gel or co-hydrogel, and the resulting mixture is provided at a place in the body of a patient where tissue regeneration or tissue replacement is intended.

In one embodiment, said tissue is selected from the group comprising skin tissue, nucleus pulposus in the intervertebral disc, cartilage tissue, synovial fluid and submucosal connective tissue in the bladder neck.

In one embodiment, said hydrogel or co-gel or co-hydrogel comprises one or more bioactive therapeutics that stimulate regenerative processes and/or modulate the immune response.

This disclosure describes for the first time the ability of ultrashort aliphatic macrocyclic peptides to self-assemble in water to form hydrogels. The peptide can be synthesized through a head to tail cyclization reaction either in solution after the peptide is cleaved from the resin, or directly on the resin support. The cyclic peptide is designed with alternated L and D amino acids in order to allow for efficient stacking of the single rings to form nano tubs or nano containers. The cyclic peptides in this disclosure present the first example of a cyclic hexa-peptide made entirely of a-amino acids that is able to form hydrogels.

Hydrogels made of aliphatic cyclic peptides of the present invention can form nanotubes or nanocontainers, which are able to encapsulate active compounds through non-covalent interaction. This allows for an active compound to have a protective shell that can significantly reduce degradation, for example, enzymatically. As a result, biological active compounds can be delivered over a longer period. In other words, the self-assembling cyclic peptides can function as a “Trojan horse”.

Furthermore, the nanotubes formed can be used for templating metal/ceramic/silicate and semiconductor nanotubes, which can be applied as conductor, transformer or isolators. Hereby, the cyclic peptide of the present invention is used to template the nanowires and can be removed afterwards to obtained nanowire structures.

In addition, cyclic peptides are known as biologically active compounds. Thus, cyclic peptides of the present invention have the potential to function as β-sheet breakers or as antimicrobial compounds.

Other aspects and features of the present invention will become apparent to those skilled in the art upon review of the following description of specific embodiments of the invention in conjunction with the accompanying figures.

### BRIEF DESCRIPTION OF DRAWINGS

Embodiments of the invention will now be described by way of example with reference to the accompanying drawings.

FIG. 1. Proposed self-assembling of cyclic peptides.

FIG. 2. Cyclization reaction.

(A) Scheme showing the cyclization reaction of LS6 in solution.

(B) Scheme showing solid phase cyclization of LK6.

FIG. 3. Hydrogels of cLK6 at 5 mg/mL in water and at 5 mg/mL in 1× PBS.

FIG. 4. FESEM pictures of cLS6 at two different magnifications.

FIG. 5. ¹H-NMR spectrum of cLS₆.

FIG. 6. ¹³C-NMR spectrum of cLS₆.

FIG. 7. ESI-MS spectrum of cLS₆.

FIG. 8. ¹H-NMR spectrum of cLK₆.

FIG. 9. ¹³C-NMR spectrum of cLK₆.

FIG. 10. ESI-MS spectrum of cLK₆.

Other arrangements of the invention are possible and, consequently, the accompanying drawings are not to be understood as superseding the generality of the preceding description of the invention.

### DETAILED DESCRIPTION

We have previously described ultrashort peptide sequences (3-7 residues) which have an innate tendency to self-assemble into helical fibers that ultimately result in hydrogel formation, see e.g. WO 2011/123061, US 2014/0093473 A1, WO 2014/104981 A1 of the inventors, and Hauser et al. (2011), Mishra et al. (2011).

The microarchitecture of these nanofibrous hydrogels resemble extracellular matrix, opening avenues for widespread applications as biomimetic scaffolds for tissue engineering and three-dimensional cell culture. Furthermore, the ultrashort peptide hydrogels demonstrate remarkable mechanical stiffness, thermostability, biocompatibility, in vitro and in vivo stability. In particular, the stability of these hydrogels offer attractive advantages to applications such as developing injectable therapies for degenerative disc disease and other tissue engineering applications requiring the construct to provide structural support over long durations.

However, in developing these hydrogels for shorter-term applications, such as injectable matrices for drug and gene delivery, it is desirable to precisely control the drug release rate. However, when a co-hydrogel, containing a bioactive compound and the peptide was formulated, only a burst release could be observed, a sustained release was never achieved.

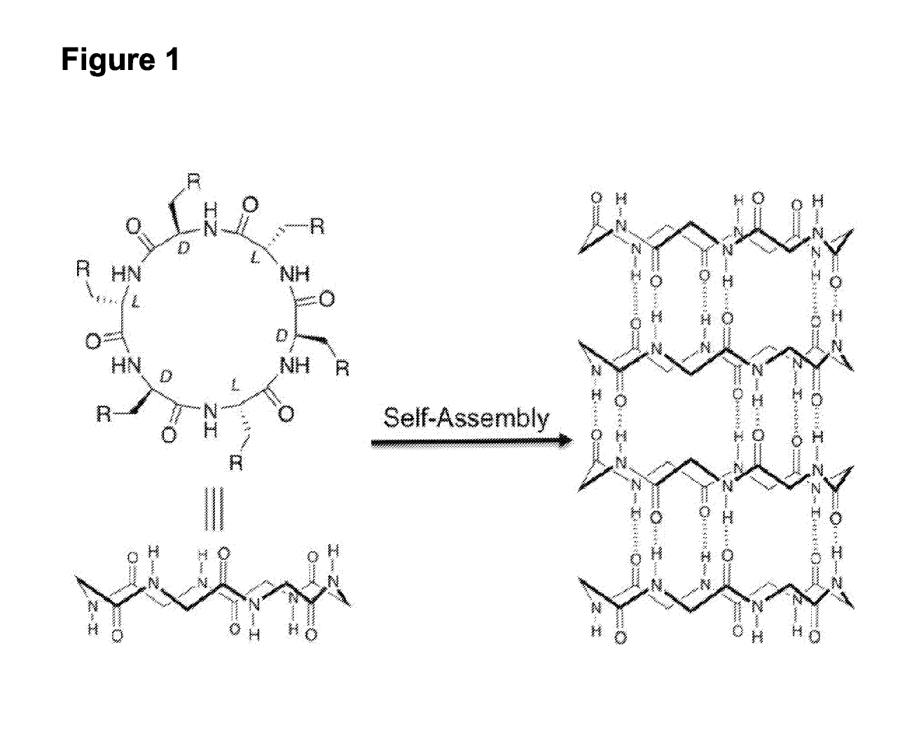

This application describes a novel class of self-assembling aliphatic cyclic peptides. Inspired by the structure of previously mentioned class of ultrashort self-assembling peptides, the cyclic peptides represent a head to tail macrocylized form of these peptides. However, to achieve self-assembly of cyclic peptide, the peptide contains alternate L-and D-amino acids (with regards to the absolute configuration, FIG. 1). In comparison to this, the parent peptides only contain one amino acid stereo isomer (all L or all D).

Although the self-assembling properties of cyclic peptides are well known (Mandal et al., 2014; Montenegro et al., 2013; Li et al., 2012), most of the reported systems do not form hydrogels. Hydrogel formation could this far only be achieved using rigid structures. Recently several groups independently report on the hydrogel formation using functionalized cyclic dipeptides. However, a cyclic dipeptide not only represents the smallest possible cyclic peptide, but is often better described as a diketopiperazine unit, and can thus be not considered as a macrocyclic peptide. Gelation of diketopiperazine is achieved through additional functionalization of the amino acid side chain and cannot be seen as an intrinsic molecular behavior (Manchineella and Govindaraju, 2012; Hoshizawa et al., 2013; Kleinsmann and Nachtsheim, 2013). To the best of our knowledge no macrocyclic peptide which can self-assemble to form hydrogels is reported this far.

In this disclosure we describe the synthesis of macrocyclic peptides which can self-assemble in water to form hydrogels made of nano-tubular fibres. These peptides are made entirely of aliphatic α-amino acids, and self-assembly is only achieved through non-covalent interaction.

### EXAMPLES

1. Materials and Methods

1.1 Materials

All Fmoc protected amino acids, O-(B enzotriazol-1-yl)-N,N,N′,N′-tetramethyluronium tetrafluoroborate (TBTU), benzotriazol-1-yl-oxytripyrrolidinophosphonium hexafluorophosphate (PyBOP) were purchased from GL Biochem (Shanghai) Ltd. Dimethylformamide (DMF) (analytical grade) was purchased from Fisher Scientific UK. Acetic anhydride (Ac₂O) and dimethyl sulfoxide (DMSO) was purchased from Sigma Aldrich. N,N-Diisopropylethylamine (DIPEA), dichloromethane (DCM), trifluoroacetic acid (TFA) and TIS (triisopropylsilane) were purchased from Alfa Aesar, a Johnson Matthey Company. Piperidine was purchased from Merck Schuchardt OHG. Diethyl ether (Et₂O) was purchased from Tedia Company Inc. All chemicals were used as received.

All peptide based compounds were purified on an Agilent 1260 Infinity preparative HPLC system equipped with a phenomenex Lunar C18 column (150×21.2 mm 5 μM). The HPLC was coupled over an active splitter to a SQ-MS for mass triggered fraction collection. MilliQ water and HPLC grade acetonitrile, both containing 0.1% formic acid, were used as eluents. ¹H and ¹³C NMR spectra were recorded on a Bruker AV-400 (400 MHz) instrument and all signals were referenced to the solvent residual peak.

1.2 Cyclic Peptide Preparation

A) Synthesis of cLS6 (cLIVAGS):

H-LIVAGS-OH was synthesized on Wang resin (GL Biochem) using SPPS following standard peptide synthesis protocols (Kirin et al., 2007). The de-protection of Fmoc was achieved by treating the resin with piperidine in DMF. The supernatant was filtered off and the resin washed with DMF. Coupling of the appropriate Fmoc-protected amino acid to the resin was done by treating the resin with a combined solution of the amino acid (3 equivalent), TBTU (3 equivalent) and DIPEA (3 equivalent) in DMF. The filtering-cum-washing, de-protection, and coupling cycle was then repeated until all the amino acids of the peptide were linked. The Fmoc deprotected peptide was cleaved from the resin using a mixture of TFA/H20/TIS (95:2.5:2.5). After precipitation with Et₂O the solid was collected by centrifugation washed with Et₂O and dried. Cyclization was carried out in solution at a concentration of 0.5 mg/mL in DMF using a threefold access of TBTU and DIPEA. The cyclization reaction was followed by HPLC-MS and if required, more coupling reagent was added to achieve full cyclization. Afterwards the solvent was removed, and the product was purified by HPLC-MS.

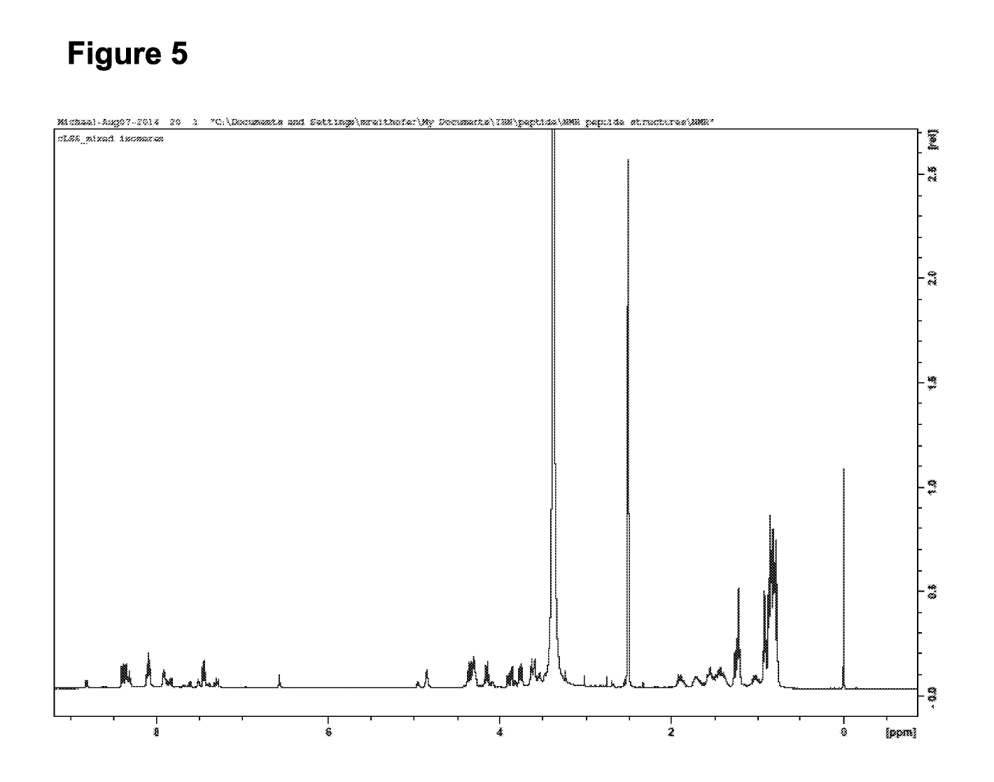

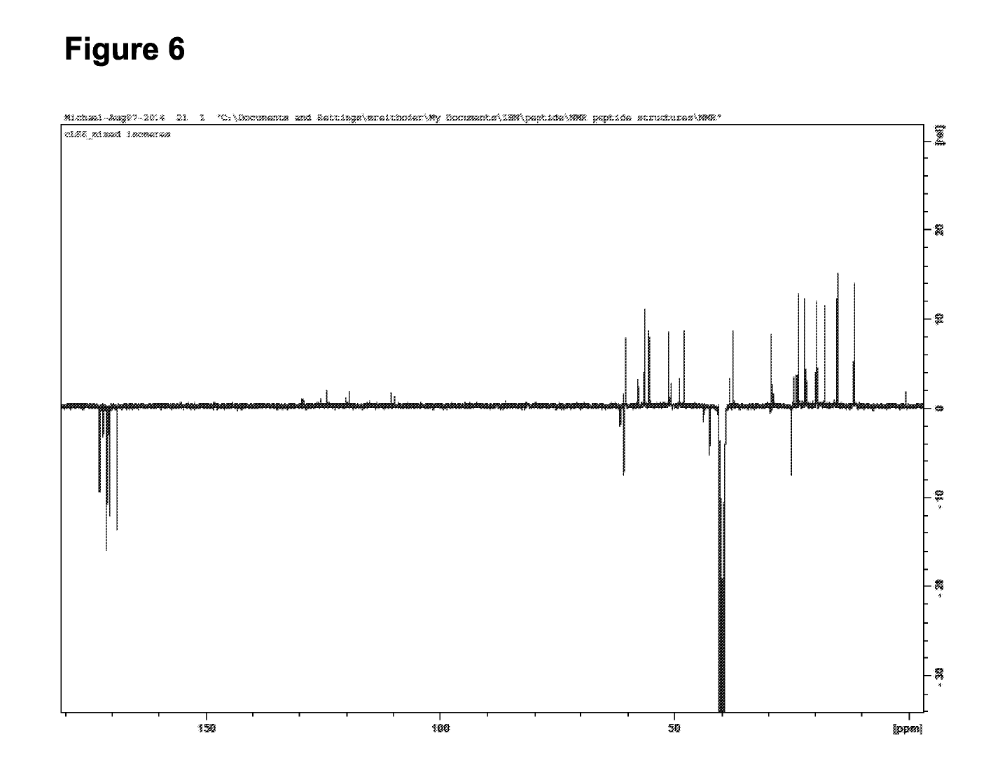

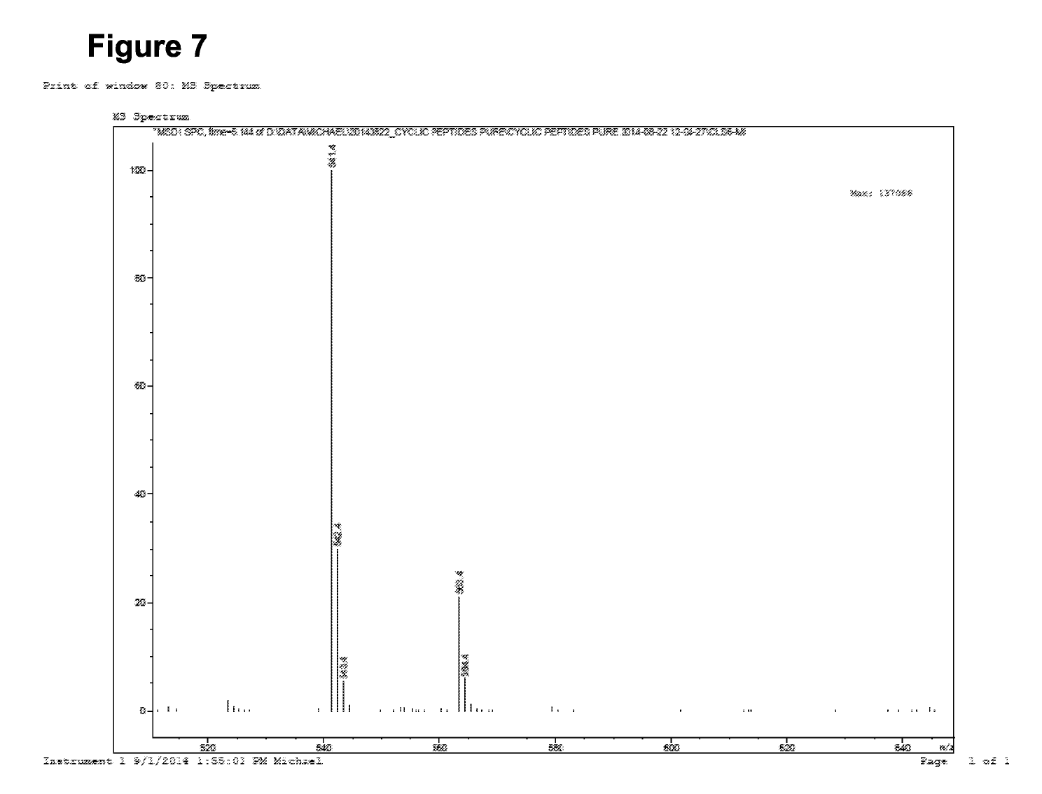

See FIGS. 5 to 7 for NMR and ESI-MS spectra.

B) Synthesis of cLK6 (cLIVAGK):

cLK6 was synthesized using standard solid phase cyclization reactions procedure (Abbour and Baudy-Floc′h, 2013). In short: Fmoc-Lys-Oallyl (1.05 mmol) was coupled to 2-chlorotrityl resin (2.1 g) in DMF/CH2Cl2 (1:3). For this purpose, CTC resin was washed once with CH₂Cl₂, afterwards, Fmoc-Lys-Oallyl, dissolved in DMF and CH₂Cl₂ was added followed by 5 equivalents of DIPEA. After 5 min an additional equivalent of DIPEA was added. The reaction was allowed to proceed for 30 min. Afterwards, the resin was quenched with MeOH to avoid side reactions. The following peptide was synthesized as described above. After Fmoc-D-Leu-OH was added, the allyl group was removed using Pd(PPh4)4 (0.1 mmol) and 10 equivalents of PhSiH₃. The reaction was allowed to proceed in CH2Cl2 in an open vessel for 1 hours. HPLC-MS confirmed full deprodection. Afterwards, the resin was washed 5 times with DMF followed by Fmoc deprodection. Final cyclization was carried out in DMF on the resin using PyBOP (4 equiv.), HOAt (4 equiv.) and DIPEA (4 equiv.) as coupling reagent. Small amounts of resin were cleaved to follow the reaction by HPLC-MS. Once complete cyclization was achieved, the peptide was cleaved from the resin as described above. After purification by HPLC-MS the pure product was obtained by lyophilization.

Yield: 160 mg (of 2.1 g resin used)

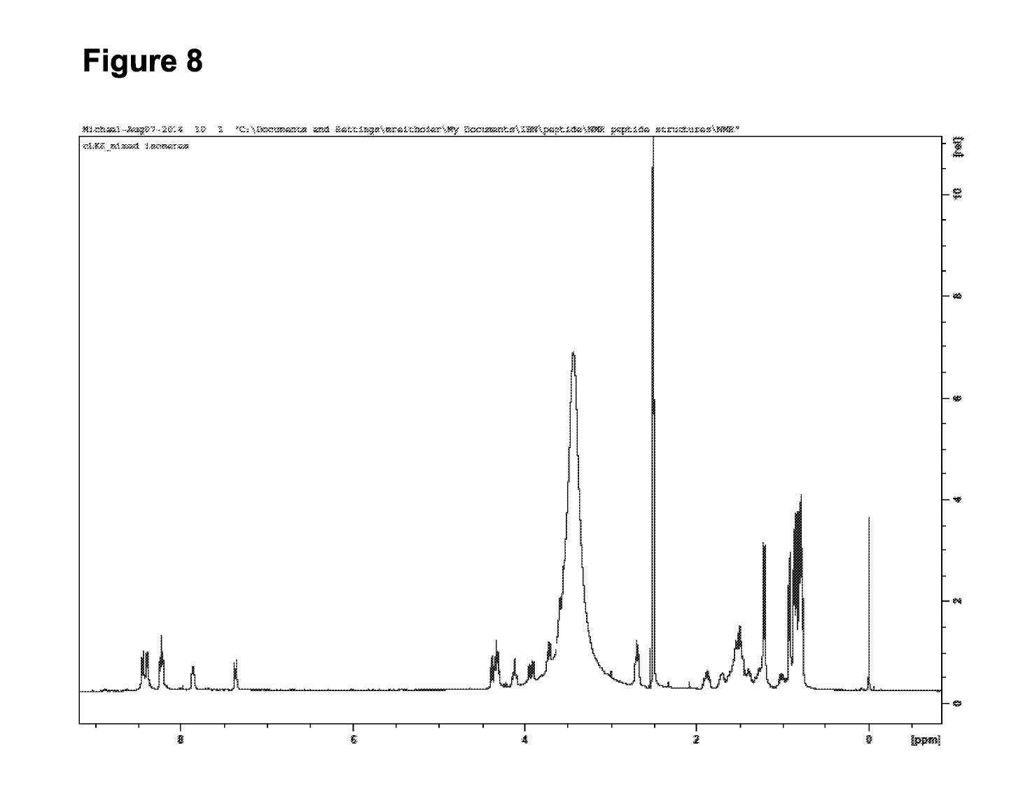

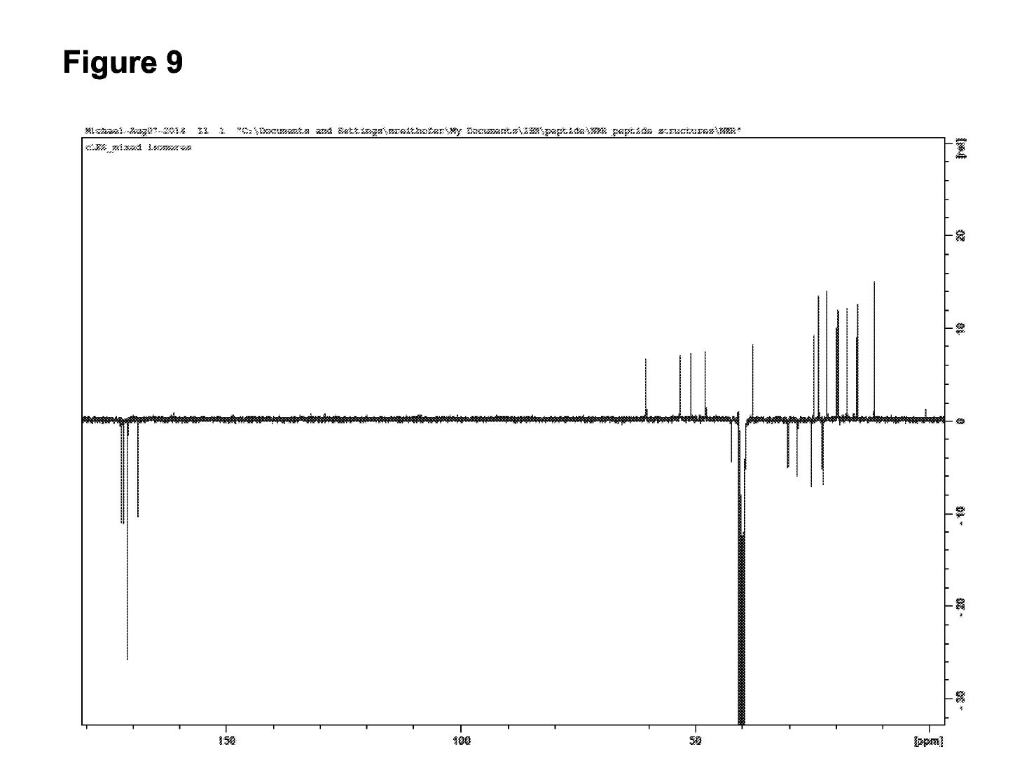

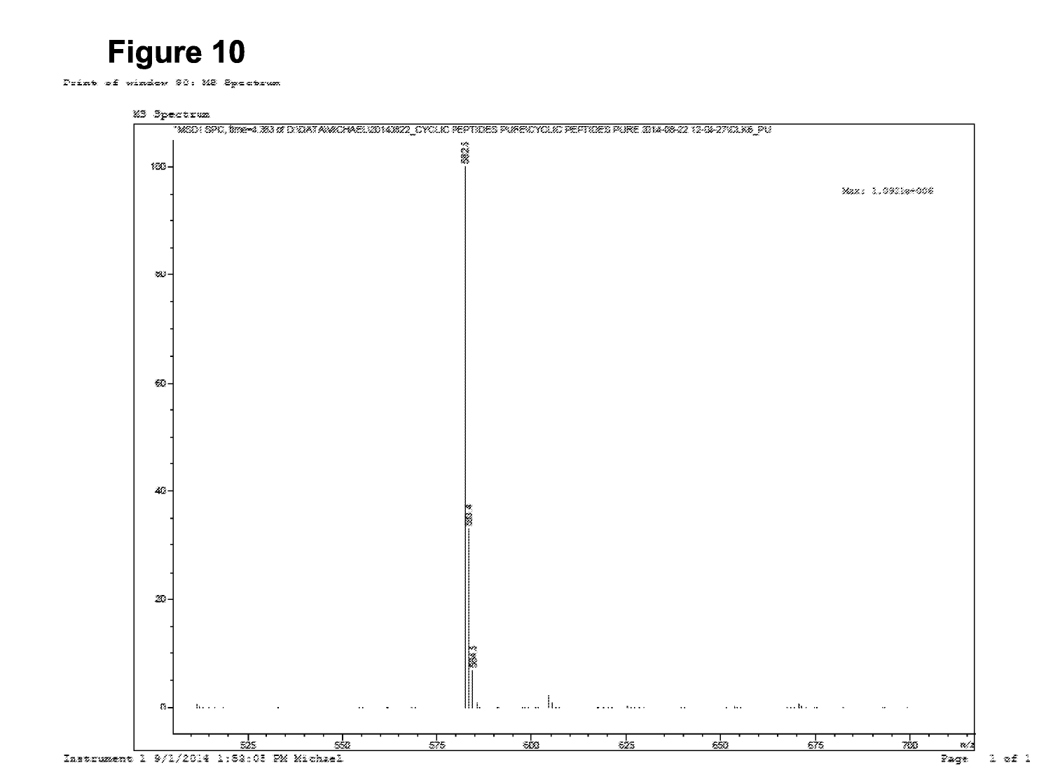

See FIGS. 8 to 10 for NMR and ESI-MS spectra.

1.3 FESEM

Hydrogel samples were shock frozen and kept at −80° C. Frozen samples were then freeze-dried. Lyophilized samples were fixed onto a sample holder using a carbon conductive tape and sputtered with platinum from both the top and the sides in a JEOL JFC-1600 High Resolution Sputter Coater. The coating current was 20 mA and the process lasted for 50 sec. The surface of interest was then examined with a JEOL JSM-7400F Field Emission Scanning Electron Microscopy (FESEM) system using an accelerating voltage of 2 kV.

2. Results and Discussion

2.1 Design and Synthesis

As discussed above, we have previously reported a new class of aliphatic amphiphilic ultrashort peptides which have an innate tendency to self-assemble in water to form biomimetic, nanofibrous hydrogels with very high mechanical strength and are extremely stable in vitro and in vivo.

In this patent application, we explore the possibility of conduction a head to tail macro cyclization reaction to obtain cyclic peptides. To achive this goal, the previously reported peptides sequences, which have been proven to form hydrogels, can be cyclized. However, to facilitate self-assembly of cyclic peptides a peptide containing alternate absolute stereo configurations of the amino acids have to be synthesized (FIG. 1).

Two parent peptide sequences were chosen to conduct a proof of concept study:

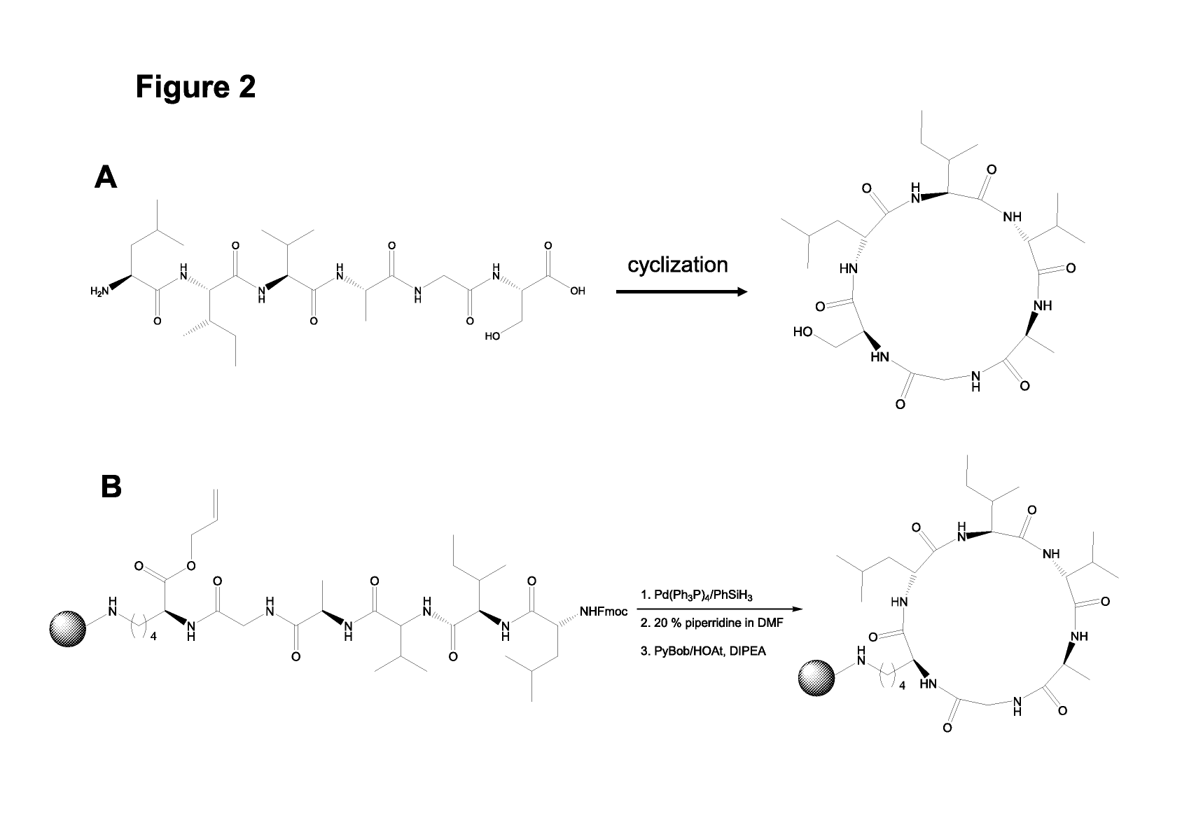

Firstly, Ac-LIVAGS-OH [SEQ ID NO. 21] was used, since it can be cyclized in solution as an unprotected peptide. For this purpose H₂N-LIVAGS-OH was synthesized by standard Fmoc-solid phase peptide synthesis (see above for details), whereby Leucine and Valine was used in D-absolute configuration. It has to be noted, that Glycine does not have a stereocenter and thus no L or D stereoisomer exists. Cyclization of H₂N-LIVAGS-OH was performed in solution using standard reaction conditions yielding cLIVAGS (=cLS₆). See FIG. 2A.

Since solution was cyclization resulted in low yield, the cyclic analog of Ac-LIVAGK-NH₂ [SEQ ID NO. 16] was synthesized entirely on the solid phase. For this purpose, an orthogonal synthetic approach was used, whereby Fmoc-Lys-OAllyl was the starting amino acid. After the entire Fmoc protected peptide was synthesized, the allyl protection group can be removed without cleaving the peptide from the resin. This allows that the final cyclization reaction is carried on solid phase and the cyclic peptide, cLIVAGK (=cLK₆) can be cleaved from the resin and purified by HPLC-MS (see above). See FIG. 2B.

2.2 Gelation Properties

In order to determine the minimum gelation concentration in water, the cyclic peptides were attempted to be dissolved in MilliQ water. As cLS6 displayed a low solubility in water, the minimum gelation concentration could not be determined. However, to prove, that cLS6 is able to self-assemble in water, cLS6 was dissolved in hexafluoroisopropanol (HFIP) and dropped slowly into water. A gelatinous “precipitate” is formed when cLS6 is dropped into water proving the ability of the cyclic peptide to self-assemble in water. The low solubility of cLS6 in water can be attributed to the absence of a charged amino acid residue.

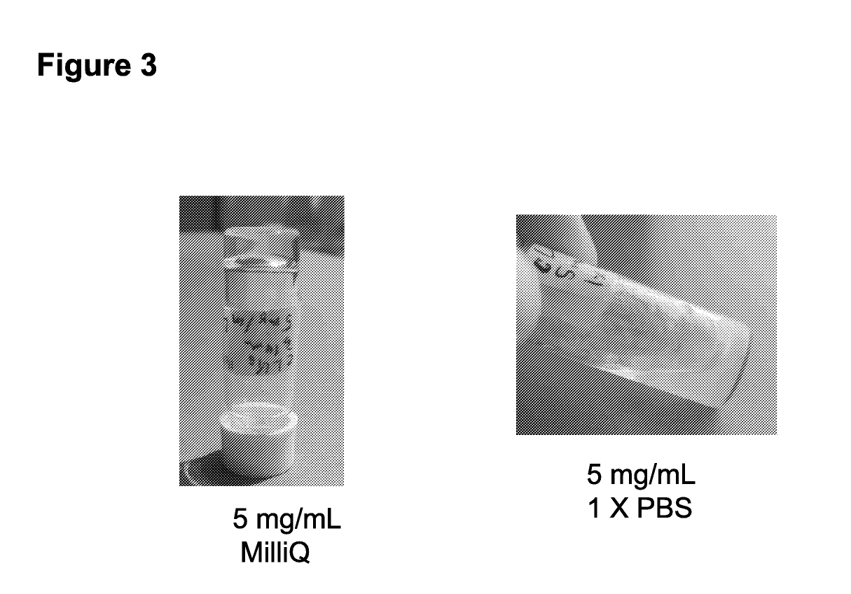

In order to introduce a charged amino acid residue cLK6 was synthesized and its ability to form hydrogels was investigated. For this purpose cLK6 was dissolved at a concentration of 10 mg/mL in water. However, full solubility was only achieved, when the peptide solution was heated at 60° C. for about 2 h. After standing at room temperature, an opaque sol gel was formed. In contrast, when a 5 mg/mL solution of cLK6 was prepared in the same way, a clear hydrogel was formed overnight (see FIG. 3). Further reduction of the peptide concentration only resulted in an increase in viscosity, but no hydrogel formation could be observed.

Our previous studies on the parent peptide Ac-LIVAGK-NH2 have shown stimuli responsive behaviour to salt, which allows to reduce the minimum gelation concentration by 50%. To test, whether cLK6 displays stimuli response to salt concentration, a 5 mg/mL 1× PBS solution was prepared. For this purpose, cLK6 was dissolved in 9 parts of water and afterwards 1 part of 10× PBS solution was added. After vortexing, only peptide aggregation, resulting in precipitation of cLK6 was observed (FIG. 3).

2.3 FESEM Study

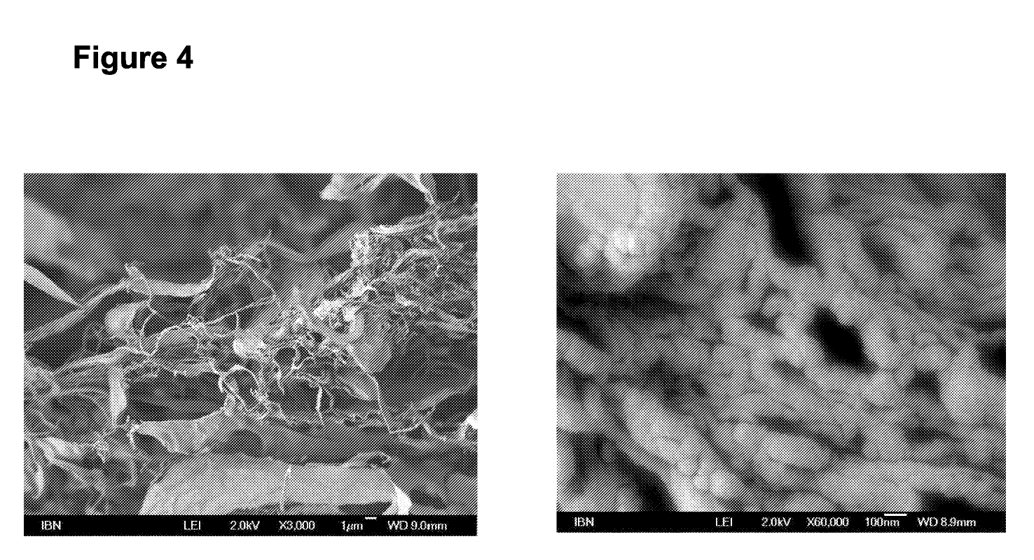

Morphological characterization of the cLS₆ hydrogel scaffolds was done by Field Emission Scanning Electron Microscopy (FESEM) and representative images are shown in FIG. 4. A fibrillization of cLS₆ is clearly visible in both images, confirming the ability of the compound to self-assemble in water.

2.4 Conclusion

We report here the synthesis of two cyclic peptides which are derived from a class of ultrashort aliphatic peptides. The cyclic peptides were synthesized though a head to tail cyclization reaction, either in solution or on solid support. Although one example, cLS6 displays limited water solubility, the compounds still displays self-assembling properties, when a solution of cLS6 dissolved in HFIP is added drop wise to water. To increase the water solubility cLK6 was synthesized, whereby the lysine residue bares a positive charge, which should increase the water solubility. Upon solubilizing cLK6 in water at 60° C. a hydrogel is formed after about 2 h standing at room temperature an opaque sol gel was formed. In contrast, when a 5 mg/mL solution of cLK6 was prepared in the same way, a clear hydrogel was formed overnight. Further reduction of the peptide concentration only resulted in an increase in viscosity, but no hydrogel formation could be observed. FESEM studies of cLS6 confirmed a fibre structure of the hydrogels proving its ability to self-assemble in water. This new material can be used for drug delivery, nano printing, as nano template, for nano wires and as additive in other peptide based hydrogels.

It is to be understood that the described embodiment(s) have been provided only by way of exemplification of this invention, and that further modifications and improvements thereto, as would be apparent to persons skilled in the relevant art, are deemed to fall within the broad scope and ambit of the present invention described herein.

### REFERENCES

S. Abbour and M. Baudy-Floc'h, Tetrahedron Letters, 2013, 54, 775-778.

C. A. E. Hauser, R. Deng, A. Mishra, Y. Loo, U. Khoe, F. Zhuang, D. W. Cheong, A. Accardo, M. B. Sullivan, C. Riekel, J. Y. Ying and U. A. Hauser, Proceedings of the National Academy of Sciences, 2011, 108, 1361-1366.

H. Hoshizawa, Y. Minemura, K. Yoshikawa, M. Suzuki and K. Hanabusa, Langmuir, 2013, 29, 14666-14673.

S. I. Kirin, F. Noor, N. Metzler-Nolte and W. Mier, Journal of Chemical Education, 2007, 84, 108.

A. J. Kleinsmann and B. J. Nachtsheim, Chemical Communications, 2013, 49, 7818-7820.

L. Li, H. Zhan, P. Duan, J. Liao, J. Quan, Y. Hu, Z. Chen, J. Zhu, M. Liu, Y.-D. Wu and J. Deng, Advanced Functional Materials, 2012, 22, 3051-3056.

S. Manchineella and T. Govindaraju, RSC Advances, 2012, 2, 5539-5542.

D. Mandal, A. Nasrolahi Shirazi and K. Parang, Organic & Biomolecular Chemistry, 2014, 12, 3544-3561.

Mishra, Y. Loo, R. Deng, Y. J. Chuah, H. T. Hee, J. Y. Ying and C. A. E. Hauser, Nano Today, 2011, 6, 232-239.

J. Montenegro, M. R. Ghadiri and J. R. Granja, Accounts of Chemical Research, 2013, 46, 2955-2965.

M. R. Reithofer, K.-H. Chan, A. Lakshmanan, D. H. Lam, A. Mishra, B. Gopalan, M. Joshi, S. Wang and C. A. E. Hauser, Chemical Science 2014, 5, 625-630.

1. A cyclic peptide and/or peptidomimetic capable of self-assembling and forming a hydrogel in aqueous solutions, the cyclic peptide and/or peptidomimetic having the general formula: 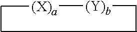
wherein
X is, at each occurrence, independently selected from the group consisting of aliphatic amino acids and aliphatic amino acid derivatives, and wherein the overall hydrophobicity decreases from N- to C-terminus;
a is an integer selected from 2 to 7;
Y is selected from the group consisting of polar amino acids and polar amino acid derivatives;
b is 0, 1 or 2;
and a+b is at least 3; and wherein all or a portion of said aliphatic amino acids and aliphatic amino acid derivatives, and said polar amino acids and polar amino acid derivatives alternate with respect to L-amino acids and D-amino acids.

2. (canceled)

3. The cyclic peptide according to claim 1, wherein said aliphatic amino acids are selected from the group consisting of alanine (Ala, A), homoallylglycine, homopropargylglycine, isoleucine (Ile, I), norleucine, leucine (Leu, L), valine (Val, V) and glycine (Gly, G), preferably from the group consisting of alanine (Ala, A), isoleucine (Ile, I), leucine (Leu, L), valine (Val, V) and glycine (Gly, G).

4. The cyclic peptide according to claim 1, wherein all or a portion of said aliphatic amino acids are arranged in an order of decreasing amino acid size, wherein the size of the aliphatic amino acids is defined as I=L>V>A>G.

5. The cyclic peptide according to claim 1, wherein (X)ₐ has a sequence selected from
(SEQ ID NO: 1)
LIVAG,
(SEQ ID NO: 2)
ILVAG,
(SEQ ID NO: 3)
LIVAA,
(SEQ ID NO: 4)
LAVAG,
(SEQ ID NO: 5)
IVAG
(SEQ ID NO: 6)
LVAG,
(SEQ ID NO: 7)
ILVA,
(SEQ ID NO: 8)
LIVA
(SEQ ID NO: 9)
LIVG
IVG,
VIG,
IVA,
VIA,
IV,
IL,
LV,
VA,
VG,
IG,
IA,
and
LA
wherein, optionally, there is an G, V or A preceding such sequence at the N-terminus, such as
(SEQ ID NO. 10)
AIVAG,
(SEQ ID NO. 11)
GIVAG,
(SEQ ID NO. 12)
VIVAG,
(SEQ ID NO. 13)
ALVAG,
(SEQ ID NO. 14)
GLVAG,
(SEQ ID NO. 15)
VLVAG.

6. The cyclic peptide according to claim 1, wherein a is an integer from 3 to 7.

7. The cyclic peptide according to claim 1, wherein said polar amino acids are selected from the group consisting of aspartic acid (Asp, D), asparagine (Asn, N), glutamic acid (Glu, E), glutamine (Gln, Q), 5-N-ethyl-glutamine (theanine), citrulline, thio-citrulline, cysteine (Cys, C), homocysteine, methionine (Met, M), ethionine, selenomethionine, telluromethionine, threonine (Thr, T), allothreonine, serine (Ser, S), homoserine, arginine (Arg, R), homoarginine, ornithine (Orn), lysine (Lys, K), N(6)-carboxymethyllysine, histidine (His, H), 2,4-diaminobutyric acid (Dab), 2,3-diaminopropionic acid (Dap), and N(6)-carboxymethyllysine.

8. The cyclic peptide according to claim 1,
wherein b is 2 and said polar amino acids are identical amino acids, or
wherein b is 1 and said polar polar amino acid comprises any one of aspartic acid, asparagine, glutamic acid, glutamine, serine, threonine, cysteine, methionine, lysine, ornithine, 2,4-diaminobutyric acid (Dab) and histidine.

9. The cyclic peptide according to claim 1, wherein (Y)_{b} has a sequence selected from Asp, Asn, Glu, Gln, Ser, Thr, Cys, Met, Lys, Orn, Dab, His, Asn-Asn, Asp-Asp, Glu-Glu, Gln-Gln, Asn-Gln, Gln-Asn, Asp-Gln, Gln-Asp, Asn-Glu, Glu-Asn, Asp-Glu, Glu-Asp, Gln-Glu, Glu-Gln, Asp-Asn, Asn-Asp Thr-Thr, Ser-Ser, Thr-Ser, Ser-Thr, Asp-Ser, Ser-Asp, Ser-Asn, Asn-Ser, Gln-Ser, Ser-Gln, Glu-Ser, Ser-Glu, Asp-Thr, Thr-Asp, Thr-Asn, Asn-Thr, Gln-Thr, Thr-Gln, Glu-Thr, Thr-Glu, Cys-Asp, Cys-Lys, Cys-Ser, Cys-Thr, Cys-Orn, Cys-Dab, Cys-Dap, Lys-Lys, Lys-Ser, Lys-Thr, Lys-Orn, Lys-Dab, Lys-Dap, Ser-Lys, Ser-Orn, Ser-Dab, Ser-Dap, Orn-Lys, Orn-Orn, Orn-Ser, Orn-Thr, Orn-Dab, Orn-Dap, Dab-Lys, Dab-Ser, Dab-Thr, Dab-Orn, Dab-Dab, Dab-Dap, Dap-Lys, Dap-Ser, Dap-Thr, Dap-Orn, Dap-Dab, Dap-Dap.

10. The cyclic peptide according to claim 1, wherein (X)ₐ-(Y)_{b} has a sequence selected from the group consisting of
(SEQ ID NO: 16)
LIVAGK,
(SEQ ID NO. 17)
ILVAGK,
(SEQ ID NO: 18)
LIVAAK,
(SEQ ID NO: 19)
LAVAGK,
(SEQ ID NO: 20)
AIVAGK,
(SEQ ID NO: 21)
LIVAGS,
(SEQ ID NO. 22)
ILVAGS,
(SEQ ID NO: 23)
LIVAAS,
(SEQ ID NO: 24)
LAVAGS,
(SEQ ID NO: 25)
AIVAGS,
(SEQ ID NO: 26)
LIVAGD,
(SEQ ID NO: 27)
ILVAGD,
(SEQ ID NO: 28)
LIVAAD,
(SEQ ID NO: 29)
LAVAGD,
(SEQ ID NO: 30)
AIVAGD,
(SEQ ID NO: 31)
LIVAGE,
(SEQ ID NO: 32)
LIVAGT,
(SEQ ID NO: 33)
ILVAGT.
(SEQ ID NO: 34)
AIVAGT,
(SEQ ID NO: 35)
AIVAGK,
(SEQ ID NO: 36)
LIVAD,
(SEQ ID NO: 37)
LIVGD,
(SEQ ID NO: 38)
IVAD,
(SEQ ID NO: 39)
IVAK,
(SEQ ID NO: 40)
LIVAGOrn,
(SEQ ID NO: 41)
ILVAGOrn,
(SEQ ID NO: 42)
AIVAGOrn,
(SEQ ID NO: 43)
LIVAGDab,
(SEQ ID NO: 44)
ILVAGDab,
(SEQ ID NO: 45)
AIVAGDab,
(SEQ ID NO: 46)
LIVAGDap,
(SEQ ID NO: 47)
ILVAGDap,
(SEQ ID NO: 48)
AIVAGDap,
(SEQ ID NO: 49)
LIVAGKK,
(SEQ ID NO: 50)
LIVAGSS,
(SEQ ID NO: 51)
LIVAGDD,
(SEQ ID NO: 52)
LIVAGEE,
IVD,
LVD,
IAK,
IVK,
LVK,
and
VAK

11. The cyclic peptide according to claim 1, wherein a+b is at least 3.

12. The cyclic peptide according to claim 1, wherein the peptides are cyclized via head-to-tail cyclization.

13. The cyclic peptide according to claim 1, wherein said cyclic peptides self-assemble in aqueous solution to form hydrogels, preferably hydrogels made of nanotubes or nanocontainers.

14. The cyclic peptide according to claim 13, wherein self-assembly is achieved through non-covalent interaction.

15.-26. (canceled)

27. A hydrogel comprising at least one cyclic peptide as defined in claim 1.

28. The hydrogel of claim 27, wherein the hydrogel is stable in aqueous solution at ambient temperature for a period of at least 1 to 6 months.

29. The hydrogel of claim 27, wherein the hydrogel is characterized by a storage modulus G′ to loss modulus G″ ratio that is greater than 2.

30. The hydrogel of claim 27, wherein the hydrogel is characterized by a storage modulus G′ from 100 Pa to 80,000 Pa at a frequency in the range of from 0.02 Hz to 16 Hz.

31. A co gel or co-hydrogel comprising
at least one cyclic peptide as defined in claim 1, and
at least one parent peptide, i.e. a peptide which has the same sequence as the cyclic peptide, but includes only L-amino acids or only D-amino acids.

32.-34. (canceled)

35. A pharmaceutical and/or cosmetic composition and/or a biomedical devive and/or a surgical implant or electronic device comprising
at least one cyclic peptide of claim 1.

36. The pharmaceutical and/or cosmetic composition and/or the biomedical device and/or the surgical implant of claim 35, further comprising a pharmaceutically active compound, and optionally a pharmaceutically acceptable carrier.

37. The pharmaceutical and/or cosmetic composition of claim 35, which is injectable.

38.-49. (canceled)


SEQUENCE LISTING (52 sequences)

SEQ ID NO: 1 - protein, 5 aa, Artificial Sequence
  (X)a of cyclic peptide
LIVAG

SEQ ID NO: 2 - protein, 5 aa, Artificial Sequence
  (X)a of cyclic peptide
ILVAG

SEQ ID NO: 3 - protein, 5 aa, Artificial Sequence
  (X)a of cyclic peptide
LIVAA

SEQ ID NO: 4 - protein, 5 aa, Artificial Sequence
  (X)a of cyclic peptide
LAVAG

SEQ ID NO: 5 - protein, 4 aa, Artificial Sequence
  (X)a of cyclic peptide
IVAG

SEQ ID NO: 6 - protein, 4 aa, Artificial Sequence
  (X)a of cyclic peptide
LVAG

SEQ ID NO: 7 - protein, 4 aa, Artificial Sequence
  (X)a of cyclic peptide
ILVA

SEQ ID NO: 8 - protein, 4 aa, Artificial Sequence
  (X)a of cyclic peptide
LIVA

SEQ ID NO: 9 - protein, 4 aa, Artificial Sequence
  (X)a of cyclic peptide
LIVG

SEQ ID NO: 10 - protein, 5 aa, Artificial Sequence
  (X)a of cyclic peptide
AIVAG

SEQ ID NO: 11 - protein, 5 aa, Artificial Sequence
  (X)a of cyclic peptide
GIVAG

SEQ ID NO: 12 - protein, 5 aa, Artificial Sequence
  (X)a of cyclic peptide
VIVAG

SEQ ID NO: 13 - protein, 5 aa, Artificial Sequence
  (X)a of cyclic peptide
ALVAG

SEQ ID NO: 14 - protein, 5 aa, Artificial Sequence
  (X)a of cyclic peptide
GLVAG

SEQ ID NO: 15 - protein, 5 aa, Artificial Sequence
  (X)a of cyclic peptide
VLVAG

SEQ ID NO: 16 - protein, 6 aa, Artificial Sequence
  (X)a-(Y)b of cyclic peptide
LIVAGK

SEQ ID NO: 17 - protein, 6 aa, Artificial Sequence
  (X)a-(Y)b of cyclic peptide
ILVAGK

SEQ ID NO: 18 - protein, 6 aa, Artificial Sequence
  (X)a-(Y)b of cyclic peptide
LIVAAK

SEQ ID NO: 19 - protein, 6 aa, Artificial Sequence
  (X)a-(Y)b of cyclic peptide
LAVAGK

SEQ ID NO: 20 - protein, 6 aa, Artificial Sequence
  (X)a-(Y)b of cyclic peptide
AIVAGK

SEQ ID NO: 21 - protein, 6 aa, Artificial Sequence
  (X)a-(Y)b of cyclic peptide
LIVAGS

SEQ ID NO: 22 - protein, 6 aa, Artificial Sequence
  (X)a-(Y)b of cyclic peptide
ILVAGS

SEQ ID NO: 23 - protein, 6 aa, Artificial Sequence
  (X)a-(Y)b of cyclic peptide
LIVAAS

SEQ ID NO: 24 - protein, 6 aa, Artificial Sequence
  (X)a-(Y)b of cyclic peptide
LAVAGS

SEQ ID NO: 25 - protein, 6 aa, Artificial Sequence
  (X)a-(Y)b of cyclic peptide
AIVAGS

SEQ ID NO: 26 - protein, 6 aa, Artificial Sequence
  (X)a-(Y)b of cyclic peptide
LIVAGD

SEQ ID NO: 27 - protein, 6 aa, Artificial Sequence
  (X)a-(Y)b of cyclic peptide
ILVAGD

SEQ ID NO: 28 - protein, 6 aa, Artificial Sequence
  (X)a-(Y)b of cyclic peptide
LIVAAD

SEQ ID NO: 29 - protein, 6 aa, Artificial Sequence
  (X)a-(Y)b of cyclic peptide
LAVAGD

SEQ ID NO: 30 - protein, 6 aa, Artificial Sequence
  (X)a-(Y)b of cyclic peptide
AIVAGD

SEQ ID NO: 31 - protein, 6 aa, Artificial Sequence
  (X)a-(Y)b of cyclic peptide
LIVAGE

SEQ ID NO: 32 - protein, 6 aa, Artificial Sequence
  (X)a-(Y)b of cyclic peptide
LIVAGT

SEQ ID NO: 33 - protein, 6 aa, Artificial Sequence
  (X)a-(Y)b of cyclic peptide
ILVAGT

SEQ ID NO: 34 - protein, 6 aa, Artificial Sequence
  (X)a-(Y)b of cyclic peptide
AIVAGT

SEQ ID NO: 35 - protein, 6 aa, Artificial Sequence
  (X)a-(Y)b of cyclic peptide
AIVAGK

SEQ ID NO: 36 - protein, 5 aa, Artificial Sequence
  (X)a-(Y)b of cyclic peptide
LIVAD

SEQ ID NO: 37 - protein, 5 aa, Artificial Sequence
  (X)a-(Y)b of cyclic peptide
LIVGD

SEQ ID NO: 38 - protein, 4 aa, Artificial Sequence
  (X)a-(Y)b of cyclic peptide
IVAD

SEQ ID NO: 39 - protein, 4 aa, Artificial Sequence
  (X)a-(Y)b of cyclic peptide
IVAK

SEQ ID NO: 40 - protein, 6 aa, Artificial Sequence
  (X)a-(Y)b of cyclic peptide wherein Xaa = ornithine (Orn)
  misc_feature (6)..(6): Xaa can be any naturally occurring amino acid
LIVAGX

SEQ ID NO: 41 - protein, 6 aa, Artificial Sequence
  (X)a-(Y)b of cyclic peptide wherein Xaa = ornithine (Orn)
  misc_feature (6)..(6): Xaa can be any naturally occurring amino acid
ILVAGX

SEQ ID NO: 42 - protein, 6 aa, Artificial Sequence
  (X)a-(Y)b of cyclic peptide wherein Xaa = ornithine (Orn)
  misc_feature (6)..(6): Xaa can be any naturally occurring amino acid
AIVAGX

SEQ ID NO: 43 - protein, 6 aa, Artificial Sequence
  (X)a-(Y)b of cyclic peptide wherein Xaa = 2,4-diaminobutyric acid (Dab)
  misc_feature (6)..(6): Xaa can be any naturally occurring amino acid
LIVAGX

SEQ ID NO: 44 - protein, 6 aa, Artificial Sequence
  (X)a-(Y)b of cyclic peptide wherein Xaa = 2,4-diaminobutyric acid (Dab)
  misc_feature (6)..(6): Xaa can be any naturally occurring amino acid
ILVAGX

SEQ ID NO: 45 - protein, 6 aa, Artificial Sequence
  (X)a-(Y)b of cyclic peptide wherein Xaa = 2,4-diaminobutyric acid (Dab)
  misc_feature (6)..(6): Xaa can be any naturally occurring amino acid
AIVAGX

SEQ ID NO: 46 - protein, 6 aa, Artificial Sequence
  (X)a-(Y)b of cyclic peptide wherein Xaa = 2,3-diaminopropionic acid (Dap)
  misc_feature (6)..(6): Xaa can be any naturally occurring amino acid
LIVAGX

SEQ ID NO: 47 - protein, 6 aa, Artificial Sequence
  (X)a-(Y)b of cyclic peptide wherein Xaa = 2,3-diaminopropionic acid (Dap)
  misc_feature (6)..(6): Xaa can be any naturally occurring amino acid
ILVAGX

SEQ ID NO: 48 - protein, 6 aa, Artificial Sequence
  (X)a-(Y)b of cyclic peptide wherein Xaa = 2,3-diaminopropionic acid (Dap)
  misc_feature (6)..(6): Xaa can be any naturally occurring amino acid
AIVAGX

SEQ ID NO: 49 - protein, 7 aa, Artificial Sequence
  (X)a-(Y)b of cyclic peptide
LIVAGKK

SEQ ID NO: 50 - protein, 7 aa, Artificial Sequence
  (X)a-(Y)b of cyclic peptide
LIVAGSS

SEQ ID NO: 51 - protein, 7 aa, Artificial Sequence
  (X)a-(Y)b of cyclic peptide
LIVAGDD

SEQ ID NO: 52 - protein, 7 aa, Artificial Sequence
  (X)a-(Y)b of cyclic peptide
LIVAGEE